(19) United States
(12) Patent Application Publication
(10) Pub. No.: US 2022/0044783 A1
(43) Pub. Date: Feb. 10, 2022

(54) Control Method and Artificial Pancreas for Administration of Insulin, Glucagon and Rescue Carbohydrates

(71) Applicant: UNIVERSITAT POLITÈCNICA DE VALÈNCIA (UPV), Valencia (ES)

(72) Inventors: Jorge BONDÍA COMPANY, Valencia (ES); José Luis DIEZ RUANO, Valencia (ES); Vanessa MOSCARDO GARCIA, Valencia (ES)

(21) Appl. No.: 16/989,150

(22) Filed: Aug. 10, 2020

(51) Int. Cl. G16H 20/17; A61M 5/172; G16H 50/30; G16H 10/40; G16H 50/70; G16H 50/50; G16H 10/60; G16H 70/40; G16H 50/20; G16H 40/67; G16H 40/40

(52) U.S. Cl. CPC G16H 20/17; A61M 5/1723; G16H 50/30; G16H 10/40; G16H 50/70; G16H 50/50; A61M 2205/50; G16H 70/40; G16H 50/20; G16H 40/67; G16H 40/40; A61M 2230/201; G16H 10/60

(57) ABSTRACT

Control method for glucose control by administrating insulin, glucagon and rescue carbohydrate includes the steps of determining a glucose reference and plasma glucose; calculating a control effort from the glucose reference and the plasma glucose; defining a first design parameter and a second design parameter being the first design parameter the relative weight between control actions and counterregulatory actions, and the second design parameter the relative weight between the counterregulatory actions; defining transfer functions representing the glycemic effect of administrating insulin, glucagon and rescue carbohydrate, normalized to unit gain; defining sensitivity factors representing the sensitiveness of a patient to insulin, glucagon and rescue carbohydrates; and calculating the rate of insulin, glucagon and rescue carbohydrate for administering by distributing the calculated control effort.

### OBJECT OF THE INVENTION

The present invention is framed in the technological field of glucose control. Specifically, the invention is directed to control actions, by the administration of hormones and/or rescue carbohydrates, to control the glucose level more effectively.

An object of the present invention is to provide a control method for administration of insulin, glucagon and rescue carbohydrates which allows to control the glucose level using in a coordinate way the three control actions.

A second object of the present invention is to provide an artificial pancreas for the administration of insulin, glucagon and rescue carbohydrates.

### BACKGROUND OF THE INVENTION

People with type 1 diabetes (T1D) lack the ability to secrete insulin and therefore are unable to regulate their blood glucose endogenously. Several technologies have been developed to maintain normoglycemia in patients by means of exogenous insulin delivery (e.g. Multiple Daily Injections -MDI- with insulin pens or Continuous Subcutaneous Insulin Infusion -CSII- with insulin pumps).

Nevertheless, achieving a good glucose control can be extremely difficult for patients due to factors like physiological variability, large glycemic impact of disturbances like exercise and meals, whose carbohydrate content must be estimated by the patient, and delays in subcutaneous insulin delivery compared to pancreatic secretion, among others.

Technological improvements in continuous glucose monitoring (CGM) have allowed its integration with insulin pumps, providing sensor-augmented systems which can modulate insulin infusion based on glucose measurements and predictions.

A predictive-low-glucose-suspend (PLGS) system automatically suspends insulin infusion when hypoglycemia risk is predicted from glucose measurements, which is resumed once the risk has been mitigated.

An artificial pancreas (AP) is an automated insulin delivery system aiming at the improvement of glucose control in people with type 1 diabetes, where, contrary to PLGS, automatic control actions are taken at each glucose measurement driven by a control algorithm.

A single-hormone AP (SHAP) consists in a continuous glucose monitor, an insulin pump and a control algorithm that modulates insulin infusion quasi-continuously.

Dual-hormone AP systems (DHAP) also introduce glucagon infusion as control action to compensate the uni-directional effect of insulin on glucose. Recent developments on stable soluble glucagon formulations such as dasiglucagon are paving the way to such systems. However, long-term safety of glucagon delivery is unknown. As well, a maximum daily dose must be established, typically 1 mg/day, to avoid side effects of glucagon like nausea, vomiting and headache.

Since demonstration of feasibility of DHAP systems in humans, several studies have targeted head-to-head comparisons between SHAP and DHAP systems. A recent review of results concluded that: during nocturnal period SHAP was enough for a good glucose control while DHAP proved superior performance in reduction of hypoglycemia overall and during exercise; benefits in post-prandial control, reduction of severe hypoglycemia and mean glucose are unclear.

In a 4-arm 4-day outpatient study with three moderate-intensity aerobic exercise sessions, DHAP achieved lower time in hypoglycemia during exercise compared to SHAP and PLGS, and similar to current standard of care where pre-exercise insulin adjustments by the patient were allowed. However, despite the use of glucagon and wearables to detect exercise, hypoglycemia was still present (1.3% mean value, or 1.0 standard deviation (SD) value, overall and 3.4% mean value, or 4.5 SD value, during exercise).

Current DHAP systems are based on an insulin controller and a glucagon controller which is activated in certain circumstances in order to initiate the counterregulatory action. These independent control loops may create unwanted interactions among hormones delivery reducing effectiveness.

Besides, an excess of plasma insulin has been found to reduce effectiveness of glucagon microboluses, which does not support the design of DHAP systems with aggressive insulin infusion considering the availability of glucagon to compensate the increased risk of hypoglycemia.

Indeed, physiologically there is a coordination between insulin and glucagon secretion, produced by the beta and alpha cells in the pancreas, respectively. On the one hand, an increment in plasma insulin levels produces a suppression of glucagon secretion in T1D patients; and a decrement in insulin levels together with low plasma glucose concentration stimulates glucagon secretion. On the other hand, alpha cells anticipate the possible hyperglycemic rebounds due to the glucagon secretion by means of beta cell sensitization.

Motivated by this paracrine communication, control algorithms incorporating coordinated insulin and glucagon delivery have been investigated. Potentiation of insulin by glucagon was incorporated in the Imperial College AP system, reporting in silico a reduction in hyperglycemia without increased hypoglycemia, compared to its non-coordinated counterpart.

In Bondia et al. a control algorithm with intrinsic insulin and glucagon coordination based on a collaborative parallel control formulation was first introduced. Thorough in silico evaluation showed the benefit of coordination with lower glucagon delivery, although room for improvement under exercise was identified.

Further refinements incorporating Sliding Mode Reference Conditioning (SMRC) techniques for insulin-on-board limitation were carried out in Moscardó. When compared to the original algorithm, the refined controller showed improvements in percentage of time in target (92.98%(3.24) vs. 91.56%(3.42)) and time in hypoglycemia (1.45%(2.01) vs. 3.40%(2.92)) in a two-week scenario with daily 60-min exercise sessions.

Nevertheless, there were some few patients that required glucagon delivery higher than 1 mg/day (0.75 mg/day [0.40; 1.83]; median[25-75 percentiles]), which may be due to a bigger impact of exercise on these patients.

### DESCRIPTION OF THE INVENTION

The present invention is related to a control method for administration of insulin, glucagon and rescue carbohydrates, which are used to compensate an excess of glucagon need in some patients. A parallel control structure incorporating automatic carbohydrate intake (DH-CC-CHO) is used.

The control method of the invention uses a main controller to compute the needed control effort (or virtual control action, Δμ(t)) and a divisor to provide an intrinsic coordination, distributing said needed control effort into different control actions or channels (Δν1(t) producing after filtering an insulin infusion (Δu(t)), Δν2(t) producing after filtering a glucagon infusion (Δw(t)), and Δν3(t) producing after filtering a rescue carbohydrates intake (Δr(t))) to get a combined effect equal to the needed control effort (Δμ(t)=Δν1(t)+Δν2(t)+Δν3(t)). Rescue carbohydrates intake (Δr(t)) is later quantized to ease administration by the patient.

The notation Δ indicates a deviation from a basal value, resulting from a process of system's linearization. Basal value is zero for all signals except for insulin delivery (Δu(t)), whose basal value is the patient's basal insulin infusion (u*), so that u(t)=Δu(t)+u*, being u(t) the insulin infusion rate sent to the insulin pump, and for glucose measurement (ΔG(t)) and glucose target (ΔGref(t)), whose basal value is the steady-state value of glucose (G*) for that basal insulin infusion (u*), so that ΔG(t)=G(t)−G* and ΔGref(t)=Gref(t)−G*, being G(t) the glucose measurement as provided by a continuous glucose monitor (CGM) and Gref(t) the glucose target as provided by the user. Glucagon infusion rate sent to the pump is w(t)=Δw(t). Carbohydrate intake prior to quantization is r(t)=Δr(t).

The main controller, expressed as C(s) in terms of its transfer function in the Laplace variable s, can be of any type. Preferably a PD controller is used, wherein the transfer function C(s) is defined as:

$$C ( S ) = \frac{Δμ ( s )}{Δ E ( s )} = K p * ( 1 + Td * s ) \qquad Eq . 1$$

In Eq. 1, Δμ(s) is the Laplace transform of the control action (Δμ(t)), ΔE(s) is the Laplace transform of the error (Δe(t)), which is defined as the difference between the target glucose (ΔGref(t)) and the measured glucose (ΔG(t)), that is, Δe(t)=ΔGref(t)−ΔG(t)=Gref(t)−G(t)=e(t). Kp represents the proportional gain and Td, the derivative time. Eq. 1 is equivalent to the time expression

$$Δ μ ( t ) = Kp * Δ e ( t ) + Kp * Td * \frac{d e ( t )}{dt} \qquad Eq . 2$$

In one embodiment, the error is filtered to avoid too large control actions for high frequency signals such as measurement noise. In this case Eq. 1 is substituted by

$$C ( s ) = \frac{Δμ ( s )}{Δ E ( s )} = Kp * ( 1 + \frac{Td * s}{1 + ( Td / N ) * s} ) \qquad Eq . 3$$

where N/Td defines the cut-off frequency of the error filter used in the computation of the derivative action, with typical values between 2 and 20. This is equivalent to the time expression:

$$\begin{matrix}Δ μ ( t ) = Kp * e ( t ) + Kp * Td * \frac{d {e}_{f} ( t )}{d t} & Eq . 4 \\ \frac{d {e}_{f} ( t )}{d t} = \frac{N}{T d} * ( - {e}_{f} ( r ) + e ( t ) ) & Eq . 5\end{matrix}$$

wherein e_{f}(t) is the filtered error, produced by said filter (Eq 5).

In another embodiment, the target glucose (ΔGref(t)) is weighted to facilitate achievement of simultaneous performance specifications under changes in glucose target (tracking) and control under disturbances like meals and exercise (disturbance rejection), giving rise to a two-degrees-of-freedom controller, with a time expression of the control action computed as:

$$Δ μ ( t ) = Kp * ( b * Δ Gref ( t ) - Δ G ( t ) ) + Kp * T d * ( c * \frac{dGref ( t )}{dt} - \frac{dG ( t )}{dt} ) \qquad Eq . 6$$

where b and c are non-negative weights to adjust according to said performance specifications. A typical value is c=0 to avoid large control actions when abrupt changes in the target ΔGref(t) happens.

In another embodiment, the control action of a PD controller in its various forms can be filtered, giving rise to a controller C(s) composed by a PD controller in series with a filter (F(s)):

C(s)=PD(s)*F(s) Eq. 7

In another embodiment, a general structure controller C(s) can be used

$$C ( s ) = k * \frac{{\prod }_{i = 1}^{m} ( s - {z}_{i} )}{{\prod }_{i = 1}^{n} ( s - {p}_{i} )} \qquad Eq . 8$$

where k is the controller gain, zi the controller zeros (values of s so that C(s)=0), and pi the controller poles (values of s so that C(s)=∞). The gain, poles and zeros fully characterize a linear controller. Expanding the numerator and denominator in Eq. 8 and applying the inverse Laplace transform, the differential equation corresponding to Eq. 8 can be computed.

The controller C(s) is designed from a single-input single-output plant, with transfer function denoted as P(s), in order to get a given closed-loop dynamics:

$$Δ G ( s ) = \frac{C ( s ) * P ( s )}{1 + C ( s ) * P ( s )} * Δ Gref ( s ) + \frac{1}{1 + C ( s ) * P ( s )} * Δ d ( s ) \qquad Eq . 9$$

where Δd(s) is the Laplace transform of a disturbance acting on glucose, such as a meal or exercise.

In one embodiment the plant P(s) is defined as the transfer function between glucagon and glucose, denoted as H2(s), normalized to unit gain so that H2(0)=1. In this case, the control effort or virtual control action Δμ(t) is defined so that

ΔG(s)=H2(s)*Δμ(s) Eq. 10

where Δμ(s) is the Laplace transform of Δμ(t).

In one embodiment, the plant P(s) is defined as the transfer function between insulin and glucose, denoted as H1(s), normalized to unit gain so that H1(0)=1. In this case, the control effort or virtual control action Δμ(t) is defined so that

ΔG(s)=H1(s)*Δμ(s) Eq. 11

The present invention allows to overcome a saturation problem produced by the limitation of administering glucagon above 1 mg per day, which could cause nausea, vomiting and headache. Therefore, the control method of the invention adds a new control action (Δν3) for avoiding the stated saturation problem.

The control method of the invention provides the control effort for hypoglycemia mitigation using insulin and glucagon, thus, administering glucagon when the insulin infusion (u(t)) is lower than a threshold (uₜₕ), for example 75% of basal insulin infusion (u*). Instead of that, a dual-hormone artificial pancreas, commonly, provides the control effort for hypoglycemia mitigation when the glucose level is low or hypoglycemia is expected. The control method of the invention allows to replace the administration of glucagon (w(t)) when the 1 mg per day dose is not enough.

The control method of the invention comprises a step of determining an original glucose reference (ΔG_{ref}(t)=G_{ref}(t)−G*) and measured glucose (ΔG(t)=G(t)−G*). Then, using the original glucose reference (ΔG_{ref}(t)) and the measured glucose (ΔG(t)), a control effort (Δμ) is calculated so as to achieve the closed loop dynamics in Eq. 9, targeting low postprandial glucose peaks and hypoglycemia avoidance in a patient.

The control method of the invention also comprises a step of defining a first design parameter (γ1(t)) and a second design parameter (γ2(t)). These design parameters allow to control the distribution of the three control actions or channels (Δν1(t), Δν2(t) and Δν3(t)), being the first design parameter (γ1(t)) the relative weight between control actions (Δν1(t)) and counterregulatory actions (Δν2(t) and Δν3(t)), and the second design parameter (γ2(t)) the relative weight between the counterregulatory actions (Δν2(t) and Δν3(t)) defining the degree of collaboration between them. The value of these design parameters (γ1(t) and γ2(t)) are set at each time a control action computation is performed when a new glucose measurement is available (that is, the sampling time, usually every 5 minutes) to 0 or 1 according to a distribution logics, such as the one explained in the next paragraphs. The resulting time-varying distribution among control actions (Δν1(t), Δν2(t) and Δν3(t)) do not affect the closed-loop dynamics defined during the design of the controller C(s), which is stable by design conditions on C(s).

Preferably, the first design parameter (γ1(t)) is 1 when a total control effort insulin infusion (u(t)) is below a certain threshold (uₜₕ) and otherwise is 0. The total control effort insulin infusion (u(t)) is the insulin that would be infused if the total control effort was directed through the insulin infusion channel (Δν1(t)=Δμ(t)). Therefore, if it was possible to supply the entire control effort by infusing insulin (u(t)) above said threshold, then no glucagon (w(t)) or rescue carbohydrates (r(t)) would be administered.

Similarly, in one embodiment, the value of the second design parameter (γ2(t)) could be defined to be 1 when the accumulated glucagon infusion in a 24 hours-time window is less or equal than a predetermined threshold (iv), preferably 1 mg or lower, and otherwise would be 0. In this case, counterregulatory control effort is provided by rescue carbohydrates (r(t)).

In another embodiment, the value of the second design parameter (γ2(t)) could be defined to be 0, additionally to the previous case, when an estimation of plasma insulin ( [symbol] (t)), obtained by means of a pharmacokinetic model, is above a threshold (Ĭₚ) previously defined so that it is known glucagon not to be effective enough. In this case, the counterregulatory control effort is provided by rescue carbohydrates (r(t)).

An example of insulin pharmacokinetic model is given by Eq. 12-14:

$$\begin{matrix}\frac{d {\hat{S}}_{1} ( t )}{d t} = - \frac{1}{{τ}_{i}} {\hat{S}}_{1} ( t ) + u ( t ) & Eq . 12 \\ \frac{d {\hat{S}}_{2} ( t )}{d t} = - \frac{1}{{τ}_{i}} {\hat{S}}_{2} ( t ) + \frac{1}{{τ}_{i}} {\hat{S}}_{1} ( t ) & Eq . 13 \\ \frac{d {\hat{I}}_{p} ( t )}{dt} = - {k}_{e} {\hat{I}}_{p} ( t ) + \frac{1}{{τ}_{i} {V}_{i}} {\hat{S}}_{2} ( t ) & Eq . 14\end{matrix}$$

In Eq. 12-14, u(t) is the insulin infusion and Ŝ₁(t), Ŝ₂(t), and Îₚ(t) are estimates of a first and second compartment of subcutaneous insulin mass, and plasma insulin concentration, respectively. Model parameters are the subcutaneous transport time constant τᵢ, plasma insulin elimination rate kₑ, and insulin distribution volume Vᵢ. A range of values for the threshold Ĭₚ is defined as Ĭₚ=ρÎₚ*, with ρ≥1 and Îₚ* the plasma insulin concentration at equilibrium for basal insulin infusion u(t)=u*.

The control method of the invention provides a flexible control structure where different configurations combining insulin (u(t)), rescue carbohydrates (r(t)) and glucagon (w(t)) infusion can be designed by selecting the design parameters (γ1(t) and γ2(t)). This includes a standard SHAP fixing γ1(t)=0 for all t; a SHAP with rescue carbohydrates fixing γ2(t)=0 for all t; a standard DHAP fixing γ2(t)=1 for all t; and a DHAP with rescue carbohydrates setting 1 or 0 to γ1(t) and/or γ2(t) as described in the previous paragraphs.

Then, a first transfer function (H1(s)) for insulin, a second transfer function (H2(s)) for glucagon and a third transfer function (H3(s)) for rescue carbohydrate, are defined, normalized to unit gain so that H1(0)=H2(0)=H3(0)=1. Each transfer function represents the glycemic effect of administrating each hormone and/or rescue carbohydrates to a patient.

Then, a first sensitivity factor (α) for insulin, a second sensitivity factor (β) for glucagon and a third sensitivity factor (∈) for rescue carbohydrate are defined, wherein each sensitivity factor represents the sensitiveness of the patient to each hormone and/or rescue carbohydrates.

In the preferred embodiment, where the control effort or virtual control action Δμ(t) is defined according to Eq. 10, the following relationships are defined between the amount of insulin (Δu(t)), glucagon (Δw(t)) and rescue carbohydrate (Δr(t)) for administering. The relationships being, for constant γ1 and γ2, in terms of Laplace transforms:

$$\begin{matrix}Δ u ( s ) = \frac{1}{α} * {( \frac{H 1 ( s )}{H 2 ( s )} )}^{- 1} * ( 1 - γ 1 ) * Δ μ ( s ) & Eq . 15 \\ Δ w ( s ) = \frac{1}{β} * γ 1 * γ 2 * Δ μ ( s ) & Eq . 16 \\ Δ r ( s ) = \frac{1}{ϵ} * {( \frac{H 3 ( s )}{H 2 ( s )} )}^{- 1} * γ 1 * ( 1 - γ 2 ) * Δ μ ( s ) & Eq . 17\end{matrix}$$

In one embodiment, where the control effort or virtual control action Δμ(t) is defined according to Eq. 11, the following relationships are defined between the amount of insulin (Δu(t)), glucagon (Δw(t)) and rescue carbohydrate (Δr(t)) for administering. The relationships being, for constant γ1 and γ2, in terms of Laplace transforms is:

$$\begin{matrix}Δ u ( s ) = \frac{1}{α} * ( 1 - γ 1 ) * Δ μ ( s ) & Eq . 18 \\ Δ w ( s ) = \frac{1}{β} * {( \frac{H 2 ( s )}{H 1 ( s )} )}^{- 1} * γ 1 * γ 2 * Δ μ ( s ) & Eq . 19 \\ Δ r ( s ) = \frac{1}{ϵ} * {( \frac{H 3 ( s )}{H 1 ( s )} )}^{- 1} * γ 1 * ( 1 - γ 2 ) * Δ μ ( s ) & Eq . 20\end{matrix}$$

Then, insulin infusion to be sent to the pump is computed as u(t)=Δu(t)+u*. The glucagon infusion to be sent to the pump is computed as w(t)=Δw(t). The rescue carbohydrates intake is computed as r(t)=Δr(t).

The control method of the invention could also comprise a step of defining a rescue carbohydrates administration function (σ_{RCO}). This step allows to calculate, in a discrete way, the rescue carbohydrates intake. In this step, the rescue carbohydrates infusion (r(t)) needed to mitigate a potential hypoglycemic episode is accumulated, until its accumulated value reaching an umbral of rescue carbohydrates (rₘₐₓ), then, a predefined complete dose is administered, possibly exceeding the umbral rₘₐₓ to ease administration by the patient.

Being administered the complete dose of rescue carbohydrates, the rescue carbohydrates infusion (r(t)) needed to mitigate a potential hypoglycemic episode continues being accumulated until its accumulated value is equal to the predefined complete dose, then, the rescue carbohydrates infusion (r(t)) accumulated value is reset to 0. Should more rescue carbohydrates infusion (r(t)) is needed to mitigate the hypoglycemic episode, the process is repeated. Once the hypoglycemic episode has been mitigated with glucose returning to normoglycemic values, the accumulated value is reset to 0.

The invention also refers to an artificial pancreas system for insulin, glucagon and rescue carbohydrate administration. The artificial pancreas system comprises a delivery unit, for supplying insulin and glucagon and a display informing the value of rescue carbohydrates to be administered from an external source.

The artificial pancreas system also comprises a sensing unit for sensing the plasma glucose (G(t)) and a first calculation unit to carry out the steps of the control method of the invention previously described.

The first calculation unit receives a glucose reference (G_{ref}(t)), which is processed, together with the sensed plasma glucose (G(t)), by a master controller for determining a control effort (Δμ(t)).

The first calculation unit also comprises a divisor, for distributing the control effort (Δμ(t)) between three channels: Δν1(t) (insulin infusion channel), Δν2(t) (glucagon infusion channel) and Δν3(t) (rescue carbohydrates channel).

Then, a first, a second and a third conversion filters determine the amount of insulin (Δu(t)), glucagon (Δw(t)) or rescue carbohydrates (Δr(t)) to be administered after addition of basal values (u(t)=Δu(t)+u*, w(t)=Δw(t), r(t)=Δr(t)).

The artificial pancreas of the invention could also comprise a second calculation unit for calculating a discrete value of rescue carbohydrates (r(t)) to be administered following the method defined previously. In this case, the display informs about a discrete value of rescue carbohydrates to be administered.

### DESCRIPTION OF THE DRAWINGS

To complement the description being made and in order to aid towards a better understanding of the characteristics of the invention, in accordance with a preferred example of practical embodiment thereof, a set of drawings is attached as an integral part of said description wherein, with illustrative and non-limiting character, the following has been represented:

FIG. 1.—shows a schematic representation of a preferred embodiment of the artificial pancreas of the invention.

FIG. 2.—shows a block diagram of the control method for administration of insulin, glucagon and rescue carbohydrate of the invention.

FIG. 3.—shows a graph Time in target-Time in hypoglycemia in percentage for four different values of insulin infusion threshold (uₜₕ).

FIG. 4.—shows a graph Insulin per day (in units) -Glucagon per day (in mg) for four different values of insulin infusion threshold (uₜₕ).

FIG. 5.—shows the evolution of glucose concentration in mg/dL over a period of time.

FIG. 6.—shows the evolution of insulin delivery in units per minute over a period of time.

FIG. 7.—shows the evolution of glucagon delivery in mg/Kg per minute over a period of time.

FIG. 8.—shows the evolution of rescue carbohydrates in grams over a period of time.

### PREFERRED EMBODIMENT OF THE INVENTION

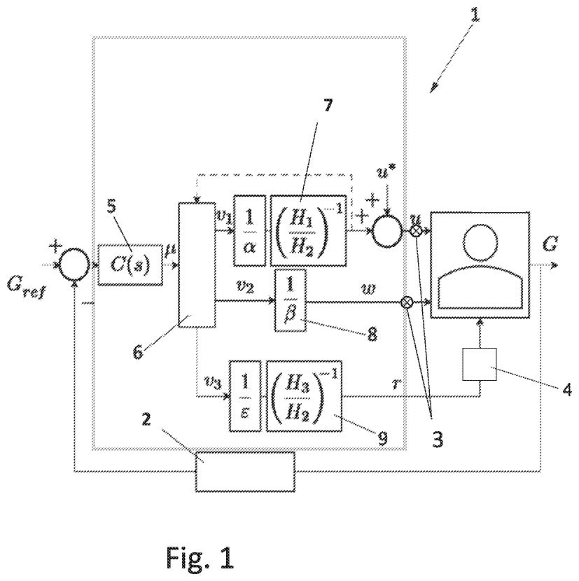

FIG. 1 represents a schematic representation of a preferred embodiment of the artificial pancreas of the invention. The artificial pancreas comprises a first calculation unit (1), a sensing unit (2) and a delivery pump (3).

The first calculation unit (1) receives a glucose reference (G_{ref}(t)) from the user, which is used by a master controller (5), together with a plasma glucose (G(t)) sensed by the sensing unit (2), so as to estimate a needed control effort (Δμ(t)). In this case, a PD controller (5) is used having as control function:

C(s)=Kp*(1+Td*s) Eq. 1

In Eq. 1, Kp represents the proportional gain and Td, the derivate time. Proportional gain Kp defines how deviations of glucose from target (the error) are weighted in the computation of the control effort. Derivative time Td and proportional gain Kp defines the weight (Kp*Td) when considering the trend of the error in the computation of the control effort. Derivative time is interpreted as the time needed for derivative and proportional actions to equal for unit error slope.

Then, a divisor (6) is used to distribute said needed control effort into different control actions or channels (Δν1(t), Δν2(t) and Δν3(t)).

The control action Δν1(t) produces after filtering an insulin infusion (Δu(t)), Δν2(t) produces after filtering a glucagon infusion (Δw(t)) and Δν3(t) produces after filtering a rescue carbohydrates infusion (Δr). Each control action is represented by means of a branch or control channel, wherein a first, a second and a third conversion units (7, 8, 9) applies a conversion function so as to determine the amount of insulin (u=Δu+u*), glucagon (w=Δw) or rescue carbohydrates (r=Δr) to be administered.

The conversion function of insulin is:

$$\frac{1}{α} * {( \frac{H 1 ( s )}{H 2 ( s )} )}^{- 1} \qquad Eq . 21$$

The conversion function of glucagon is:

$$\frac{1}{β} \qquad Eq . 22$$

The conversion function of rescue carbohydrates is:

$$\frac{1}{ɛ} * {( \frac{H 3 ( s )}{H 2 ( s )} )}^{- 1} \qquad Eq . 23$$

In Eq. 21-23, H1(s) is a first transfer function for insulin, H2(s) is a second transfer function for glucagon and H3(s) is a third transfer function for rescue carbohydrates. Each transfer function represents the glycemic effect of administrating each hormone and/or carbohydrates to a patient, normalized to unit gain (H1(0)=H2(0)=H3(0)=1).

Also, α is a first sensitivity factor for insulin, β is a second sensitivity factor for glucagon and ∈ is a third sensitivity factor for rescue carbohydrate. Each sensitivity factor represents the sensitiveness of the patient to each hormone and/or carbohydrates.

Also, a CGM branch is included, wherein is placed the sensing unit (2), which forms a loop sensing the plasma glucose after being activated the control actions, and providing an updated value to the master controller (5).

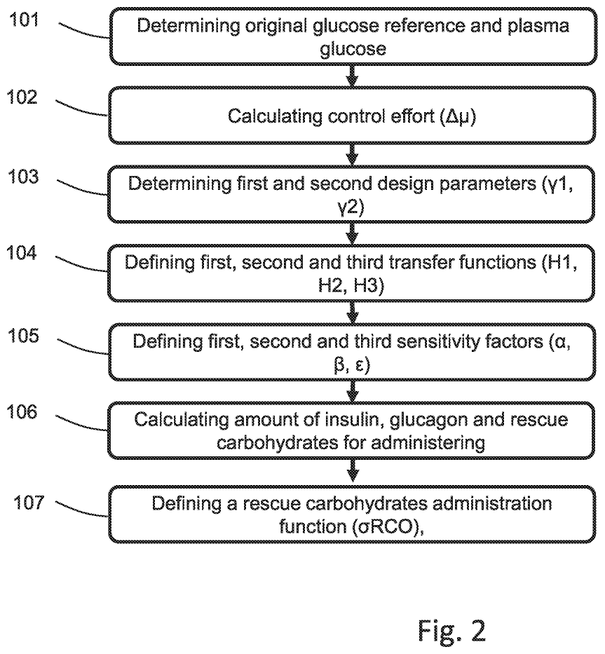

The divisor (6) distributes the control effort (Δμ(t)) according to the steps of the control method described in FIG. 2.

The control method for administration of insulin, glucagon and rescue carbohydrate of the FIG. 2 comprises a step of determining (101) a glucose reference (G_{ref}(t)), given by the user, and a plasma glucose (G(t)) by using the sensing unit (2).

Then, the glucose reference (G_{ref}(t)) is used by the master controller (5) so as to calculate (102) the control effort (Δμ(t)).

The control effort (Δμ(t)) is transferred to a divisor (6), wherein is distributed between three different control actions or channels (Δν1(t), Δν2(t) and Δν3(t)), routing the control effort through an insulin infusion channel (Δν1), glucagon infusion channel (Δν2) and rescue carbohydrates infusion channel (Δν3).

It is defined (103) a first design parameter (γ1(t)) and a second design parameter (γ2(t)). Also, it is defined (104) a first transfer function (H1(s)) for insulin, a second transfer function (H2(s)) for glucagon and a third transfer function (H3(s)) for rescue carbohydrate, wherein each transfer function represents the glycemic effect of administrating insulin, glucagon and rescue carbohydrate to a patient, normalized to unit gain (H1(0)=H2(0)=H3(0)=1).

A control effort (Δμ(t)) is defined so that the Laplace transform of the variation in plasma glucose (ΔG(s)) is equal to a selected transfer function multiplied by Laplace transform of said control effort (Δμ(s)). The selected transfer function could be any of the three transfer functions (H1(s), H2(s) or H3(s)).

Also, the variation in plasma glucose (ΔG(s)) is affected by a disturbance (Δd(s)). The disturbance represents the glycemic effect of meal and exercise. Therefore, the variation in plasma glucose (ΔG(s)) depends on the insulin and glucagon efficiency in a patient, amount of exercise, meal, and/or rescue carbohydrates taken by the patient.

Furthermore, it is defined (105) a first sensitivity factor (α) for insulin, a second sensitivity factor (β) for glucagon and a third sensitivity factor (∈) for rescue carbohydrates, wherein each sensitivity factor represents the sensitiveness of a patient to insulin, glucagon and rescue carbohydrate.

Consequently, each control action (Δν1(t), Δν2(t) and Δν3(t)) is defined, in terms of their Laplace transforms, as the correspondent sensitivity factor (α, β and ∈) multiplied by the division between the correspondent transfer function (H1(s), H2(s) and H3(s)) and the selected transfer function; multiplied by the Laplace transform of the correspondent hormone infusion and/or carbohydrate administration (Δu, Δw and Δr). Also, each control action (Δν1(t), Δν2(t) and Δν3(t)) is correlated to the control effort (Δμ(t)) following the equations:

Δν1(t)=(1−γ1(t))*Δμ(t) Eq. 24

Δν2(t)=γ1(t)*γ2(t)*Δμ(t) Eq. 25

Δν3(t)=γ1(t)*(1−γ2(t))*Δμ(t) Eq. 26

These parameters are selected so as to the value of the first design parameter (γ1(t)) is 1 when a total control effort insulin infusion (\check{u}) is below a certain insulin infusion threshold (uₜₕ), and otherwise is 0; and the value of the second design parameter (γ2(t)) is 1 when the accumulated glucagon infusion in a 24 hours-time window is less or equal than a predetermined threshold (\check{w}), and otherwise is 0.

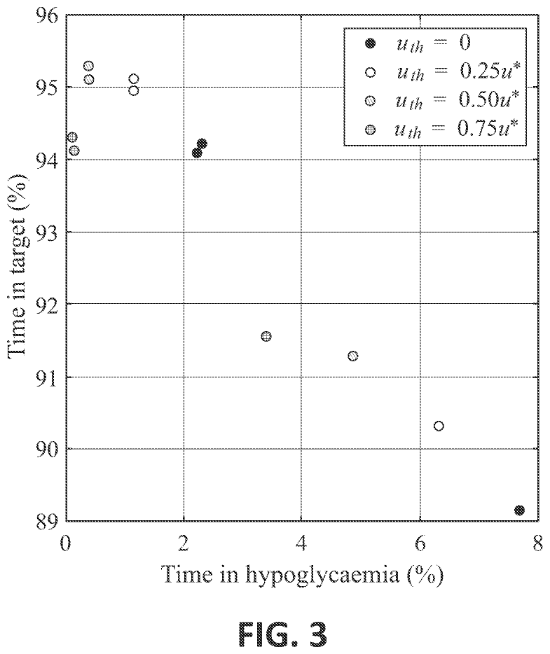

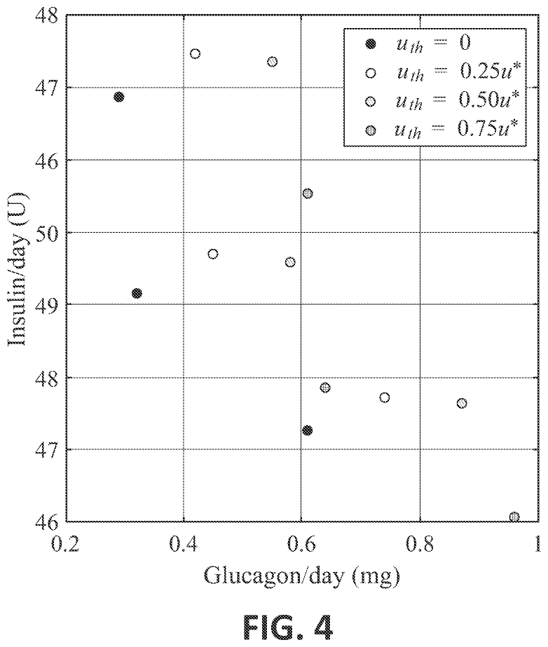

The effect of modifying the insulin infusion threshold (uₜₕ) is showed in FIGS. 3 and 4. FIG. 3 shows a graph Time in target-Time in hypoglycemia for four different values of insulin infusion threshold (uₜₕ), both in percentage. FIG. 4 shows a graph Insulin per day-Glucagon per day for the same four values of insulin infusion threshold (uₜₕ), the Insulin per day in units and the Glucagon per day in mg.

Taking into account said definitions, the divisor calculates the relationship between each control action by following the equations:

$$\begin{matrix}Δ u ( s ) = \frac{1}{α} * {( \frac{H 1 ( s )}{H 2 ( s )} )}^{- 1} * ( 1 - γ 1 ) * Δ μ ( s ) & Eq . 15 \\ Δ w ( s ) = \frac{1}{β} * γ 1 * γ 2 * Δ μ ( s ) & Eq . 16 \\ Δ r ( s ) = \frac{1}{ϵ} * {( \frac{H 3 ( s )}{H 2 ( s )} )}^{- 1} * γ 1 * ( 1 - γ 2 ) * Δ μ ( s ) & Eq . 17\end{matrix}$$

From Eq. 15-17 it is calculated (106) the amount of insulin (Δu), glucagon (Δw) and rescue carbohydrate (Δr) for administering.

In the same way, a rescue carbohydrates administration function (σ_{RCO}) is defined (109). This function allows to calculate, in a discrete way, the rescue carbohydrates intake, by means of a second calculation unit (4). Therefore, the rescue carbohydrates infusion (r(t)) needed to mitigate a potential hypoglycemic episode is accumulated, until its accumulated value reaching an umbral of rescue carbohydrates (rₘₐₓ), set as a half of a predefined complete dose which in this case is defined as 15 g. Then, the predefined complete dose is administered.

After being administered the complete dose of rescue carbohydrates, the rescue carbohydrates infusion (r(t)) needed to mitigate a potential hypoglycemic episode continues being accumulated until its accumulated value is equal to the predefined complete dose, then, the rescue carbohydrates infusion (r(t)) accumulated value is reset to 0.

In case that more rescue carbohydrates infusion (r(t)) is needed to mitigate the hypoglycemic episode, the process is repeated, and once the hypoglycemic episode has been mitigated with glucose returning to normoglycemic values, the accumulated value is reset to 0.

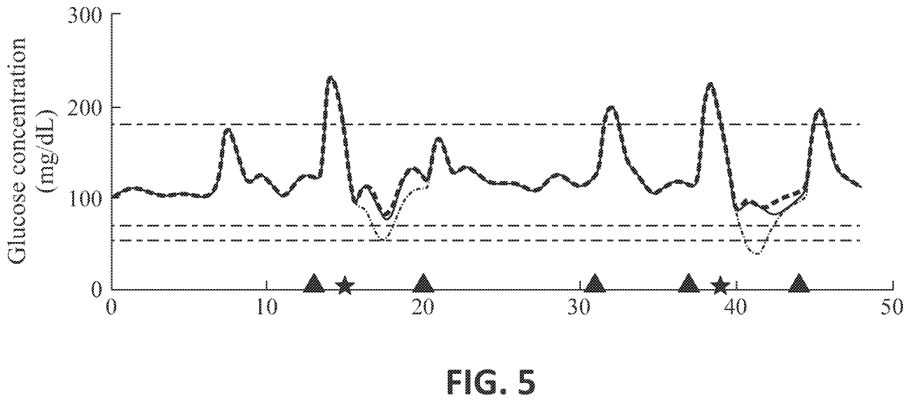

FIGS. 5 to 8 show the comparison between the control method of the invention, a DH-CC and a DH-CC having an administration of glucagon less than 1 mg. Meal events are marked with triangles and exercise events are marked with stars.

FIG. 5 shows the evolution of glucose concentration in mg/dL over a period of time, wherein the DH-CC having an administration of glucagon less than 1 mg has more time of hypoglycemia.

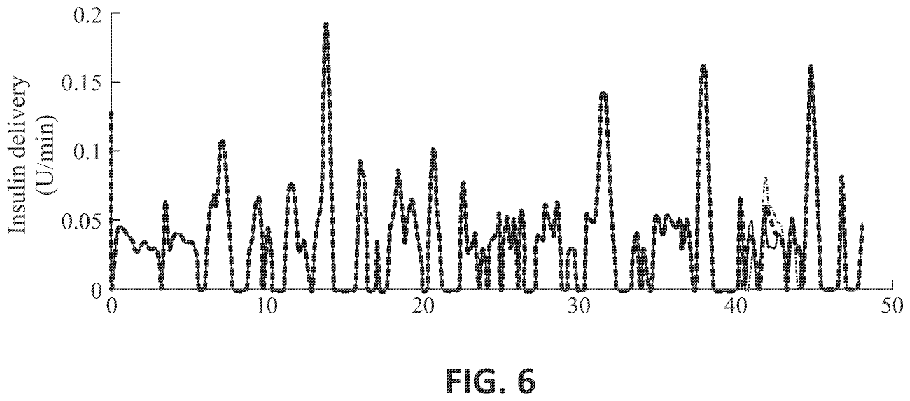

FIG. 6 shows the evolution of insulin delivery in units per minute over a period of time.

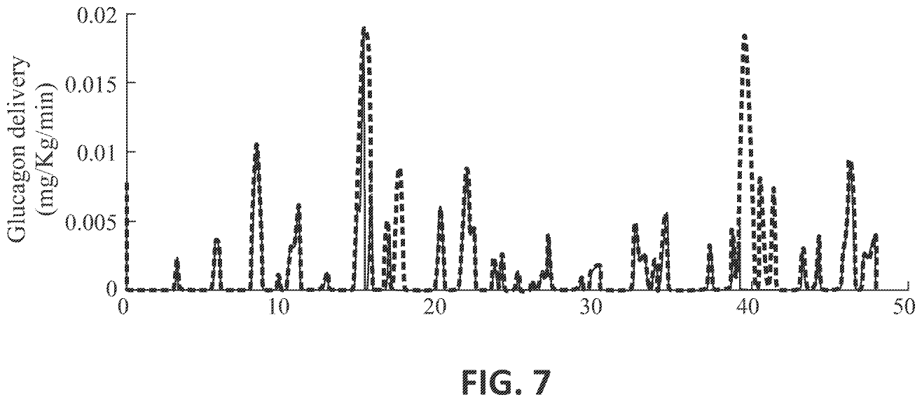

FIG. 7 shows the evolution of glucagon delivery in mg/Kg per minute over a period of time, wherein the DH-CC has more glucagon administration.

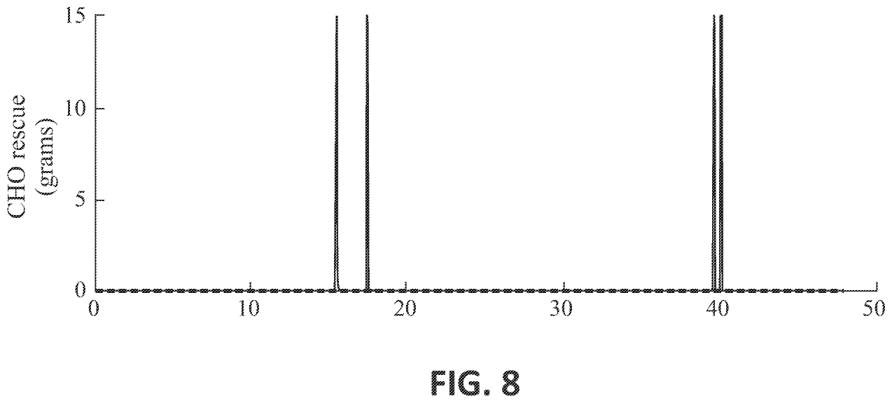

FIG. 8 shows the evolution of rescue carbohydrates in grams over a period of time, wherein only the control method of the invention administrates rescue carbohydrates.

1. A control method for administration of insulin, glucagon and rescue carbohydrate which comprises the steps of:
determining a glucose reference (G_{ref}(t)) and plasma glucose (G(t));
calculating a control effort (Δμ(t)) from the glucose reference (G_{ref}(t)) and the plasma glucose (G(t));
defining a first design parameter (γ1(t)) and a second design parameter (γ2(t)) being the first design parameter (γ1(t)) the relative weight between control actions (Δν1(t)) and counterregulatory actions (Δν2(t) and Δν3(t)), and the second design parameter (γ2(t)) the relative weight between the counterregulatory actions (Δν2(t) and Δν3(t));
defining a first transfer function (H1(s)) for insulin, a second transfer function (H2(s)) for glucagon and a third transfer function (H3(s)) for rescue carbohydrate, wherein each transfer function represents the glycemic effect of administrating insulin, glucagon and rescue carbohydrate, normalized to unit gain (H1(0)=H2(0)=H3(0)=1);
defining a first sensitivity factor (α) for insulin, a second sensitivity factor (β) for glucagon and a third sensitivity factor (∈) for rescue carbohydrate, wherein each sensitivity factor represents the sensitiveness of a patient to insulin, glucagon and rescue carbohydrate;
calculating the rate of insulin (Δu), glucagon (Δw) and rescue carbohydrate (Δr) for administering by distributing the calculated control effort (Δμ), such that the relationship between them fulfills at least one group of the following equations:
$$\begin{matrix}∘ Group 1 : & \\ Δ u ( s ) = \frac{1}{α} * {( \frac{H 1 ( s )}{H 2 ( s )} )}^{- 1} * ( 1 - γ 1 ) * Δ μ ( s ) & Eq . 15 \\ Δ w ( s ) = \frac{1}{β} * γ 1 * γ 2 * Δ μ ( s ) & Eq . 16 \\ Δ r ( s ) = \frac{1}{ϵ} * {( \frac{H 3 ( s )}{H 2 ( s )} )}^{- 1} * γ 1 * ( 1 - γ 2 ) * Δ μ ( s ) & Eq . 17 \\ ∘ Group 2 : & \\ Δ u ( s ) = \frac{1}{α} * ( 1 - γ 1 ) * Δ μ ( s ) & Eq . 18 \\ Δ w ( s ) = \frac{1}{β} * {( \frac{H 2 ( s )}{H 1 ( s )} )}^{- 1} * γ 1 * γ 2 * Δ μ ( s ) & Eq . 19 \\ Δ r ( s ) = \frac{1}{ϵ} * {( \frac{H 3 ( s )}{H 1 ( s )} )}^{- 1} * γ 1 * ( 1 - γ 2 ) * Δ μ ( s ) & Eq . 20\end{matrix}$$

2. The control method according to claim 1, wherein the value of the first design parameter (γ1(t)) is 0, resulting in a standard SHAP.

3. The control method according to claim 1, wherein the value of the second design parameter (γ2(t)) is 0, resulting in a SHAP with rescue carbohydrates.

4. The control method according to claim 1, wherein the value of the second design parameter (γ2(t)) is 1, resulting in a standard DHAP.

5. The control method according to claim 1, wherein the value of the first design parameter (γ1(t)) is 1, when a total control effort insulin infusion (\check{u}(t)), representing the insulin infusion (u(t)) when the control effort (Δμ(t)) is directed only to insulin infusion (u(t)), is below a predetermined insulin infusion threshold (uₜₕ), and otherwise is 0.

6. The control method according to claim 5, wherein the value of the second design parameter (γ2(t)) is 1 when the accumulated glucagon infusion (w(t)) in a 24 hours-time window is less or equal than a predetermined threshold (\check{w}), and otherwise is 0.

7. The control method according to claim 5, wherein the value of the second design parameter (γ2(t)) is 0 when an estimation of plasma insulin ( [symbol] (t)) obtained by means of a pharmacokinetic model is above a threshold (Ĭₚ) previously defined, so that it is known glucagon is not to be effective enough.

8. The control method according to claim 5, wherein the value of the second design parameter (γ2(t)) is 0 when an estimation of plasma insulin ( [symbol] (t)) is above a given threshold (Ĭₚ) so that it is known glucagon is not to be effective enough, and otherwise is 1.

9. The control method according to claim 1, further comprising a step of defining a rescue carbohydrates administration function (σ_{RCO}), such that, when the rescue carbohydrates infusion needed is accumulated until reaching an umbral of rescue carbohydrates (rₘₐₓ), then, a predefined complete dose is administered to ease administration by the patient, and when said accumulation of rescue carbohydrates infusion needed is equal to the predefined complete dose or once the hypoglycemic episode has been mitigated with glucose returning to normoglycemic values, the accumulated value of rescue carbohydrates infusion needed is reset to 0.

10. An artificial pancreas system for insulin, glucagon and rescue carbohydrate administration comprising:
a delivery unit, for supplying insulin and glucagon;
a sensing unit for sensing the plasma glucose (G(t));
a first calculation unit which carries out the steps of claim 1:
determining a glucose reference (G_{ref}(t)) and plasma glucose (G(t));
calculating a control effort (Δμ(t)) from the glucose reference (G_{ref}(t)) and the plasma glucose (G(t));
defining (a first design parameter (γ1(t)) and a second design parameter (γ2(t)) being the first design parameter (γ1(t)) the relative weight between control actions (Δν1(t)) and counterregulatory actions (Δν2(t) and Δν3(t)), and the second design parameter (γ2(t)) the relative weight between the counterregulatory actions (Δν3(t) and Δν3(t));
defining a first transfer function (H1(s)) for insulin, a second transfer function (H2(s)) for glucagon and a third transfer function (H3(s)) for rescue carbohydrate, wherein each transfer function represents the glycemic effect of administrating insulin, glucagon and rescue carbohydrate, normalized to unit gain (H1(0)=H2(0)=H3(0)=1);
defining a first sensitivity factor (α) for insulin, a second sensitivity factory (β) for glucagon and a third sensitivity factor (∈) for rescue carbohydrate, wherein each sensitivity factor represents the sensitiveness of a patient to insulin, glucagon and recue carbohydrate;
calculating the rate of insulin (Δu), glucagon (Δw) and rescue carbohydrate (Δr) for administering by distributing the calculated control effort (Δμ), such that the relationship between them fulfills at least one group of the following equations:
$$\begin{matrix}Group 1 : & \\ Δ u ( s ) = \frac{1}{α} * {( \frac{H 1 ( s )}{H 2 ( s )} )}^{- 1} * ( 1 - γ 1 ) * Δ μ ( s ) & Eq . 15 \\ Δ w ( s ) = \frac{1}{β} * γ 1 * γ 2 * Δ μ ( s ) & Eq . 16 \\ Δ r ( s ) = \frac{1}{ϵ} * {( \frac{H 3 ( s )}{H 2 ( s )} )}^{- 1} * γ 1 * ( 1 - γ 2 ) * Δ μ ( s ) & Eq . 17 \\ Group 2 : & \\ Δ u ( s ) = \frac{1}{α} * ( 1 - γ 1 ) * Δ μ ( s ) & Eq . 18 \\ Δ w ( s ) = \frac{1}{β} * {( \frac{H 2 ( s )}{H 1 ( s )} )}^{- 1} * γ 1 * γ 2 * Δ μ ( s ) & Eq . 19 \\ Δ r ( s ) = \frac{1}{ϵ} * {( \frac{H 3 ( s )}{H 1 ( s )} )}^{- 1} * γ 1 * ( 1 - γ 2 ) * Δ μ ( s ) & Eq . 20\end{matrix}$$
The first calculation unit comprising:
a master controller, for determining a control effort (Δμ(t)) from the plasma glucose (G(t)) and a receiving glucose reference (G_{ref} (t))
a divisor, for distributing the control effort (Δμ(t)) between three channels: insulin infusion (Δν1(t)), glucagon infusion (Δν2(t)) and rescue carbohydrates infusion (Δν3(t));
a first, a second and a third conversion units for determining the amount of insulin (u(t)), glucagon (w(t)) or rescue carbohydrates (r(t)) to be administered; and
a display informing about the value of rescue carbohydrates to be administered from an external source.

11. The artificial pancreas system according to claim 10, further comprising a second calculation unit for calculating a discrete value of rescue carbohydrates (r(t)) to be administered by carrying out the steps of defining a rescue carbohydrates administration function (σ_RCO), such that, when the rescue carbohydrates infusion needed is accumulated until reaching an umbral of rescue carbohydrates (r_max), then, a predefined complete dose is administered to ease administration by the patient, and when said accumulation of rescue carbohydrates infusion needed is equal to the predefined complete dose or once the hypoglycemic episode has been mitigated with glucose returning to normoglycemic values, the accumulated value of rescue carbohydrates infusion needed is reset to 0.
